(19) United States
(12) Patent Application Publication
(10) Pub. No.: US 2024/0341946 A1
(43) Pub. Date: Oct. 17, 2024

(54) Soft Anchor Locking Staple and Methods of Tissue Repairs

(71) Applicant: ARTHREX, INC., Naples, FL (US)

(72) Inventors: Peter J. DREYFUSS, Naples, FL (US); William T. PENNINGTON, Franklin, WI (US)

(73) Assignee: ARTHREX, INC., Naples, FL (US)

(21) Appl. No.: 18/299,806

(22) Filed: Apr. 13, 2023

(51) Int. Cl. A61F 2/08; A61B 17/064

(52) U.S. Cl. CPC A61F 2/0811; A61B 17/0642; A61B 2017/0409; A61B 2017/0445; A61F 2002/0852; A61F 2220/0075

(57) ABSTRACT

Methods for tissue repairs with soft anchor locking staples are disclosed. A locking staple is created with a surgical construct using two soft anchors connected by a fixed length of suture and without the need for any additional tensioning steps.

### FIELD OF THE INVENTION

The disclosure relates to the field of surgery and, more specifically, to anchor constructs and associated methods of tissue repairs.

### SUMMARY

Surgical constructs, assemblies, and methods for fixation of soft tissue are disclosed.

A surgical construct can create a tissue staple. A surgical construct can include two or more soft anchors connected by a fixed length of suture. The surgical construct can create a knotless, self-tensioning, self-locking, reinforced repair. The surgical construct can be employed in knotless fixation of first tissue to second tissue, for example, fixation of soft tissue to bone.

Methods of tissue repairs are also disclosed. A knotless surgical construct can provide tissue to tissue fixation without any knot formation, with fewer passing steps, with increased fixation and soft tissue compression, and without the need of any secondary tensioning steps.

### BRIEF DESCRIPTION OF THE DRAWINGS

FIG. 1 illustrates a top view of a surgical construct according to an exemplary embodiment.

FIG. 2 illustrates the surgical construct of FIG. 1 attached to an inserter.

FIG. 3 illustrates a top view of the surgical construct of FIG. 1 at a repair site.

FIG. 4 illustrates a schematic side view of the surgical construct of FIG. 1 (at a pre-insertion stage).

FIG. 5 illustrates a schematic side view of the surgical construct of FIG. 4 at a stage subsequent to that of FIG. 4 (at a post-insertion stage).

FIGS. 6-9 illustrate exemplary steps of a tissue repair with the surgical construct of FIG. 1.

FIG. 10 illustrates a top view of another tissue repair with the surgical construct of FIG. 1.

### DETAILED DESCRIPTION

Knotless, self-tensioning, self-locking surgical repair systems, assemblies, and constructs can create knotless, self-tensioning, reinforced repairs without the need for additional, secondary tensioning steps and without suture management or suture cutting steps.

A surgical construct can include a fixed length of flexible strand (fixed length of a loop) with two or more soft anchors loaded onto, and connected by, the fixed length of flexible strand. In an embodiment, two soft anchors are connected to a fixed length of flexible strand. The flexible strand forms a flexible, continuous, uninterrupted loop having a fixed length. The flexible strand can be suture. The two or more soft anchors can be all-suture knotless anchors. The surgical construct can be employed in knotless fixation of first tissue to second tissue, for example, fixation of soft tissue to bone. The surgical construct can consist essentially of a fixed length of flexible strand (fixed length of a loop) with two soft anchors.

Methods of surgical tissue repairs are also disclosed. In an embodiment, a surgical construct provides knotless first tissue to second tissue fixation, without secondary tensioning steps, without any knot formation, with fewer passing steps, and with increased fixation and soft tissue compression. A surgical construct includes two soft anchors connected by a fixed length of a flexible coupler (a fixed length of suture loop). The surgical construct is inserted into tissue in a simple manner, to lock the construct into place and to tension the construct at the time of insertion, without necessitating any additional tensioning steps. The surgical construct creates a soft anchor locking “staple” constructed of soft anchors connected by the flexible coupler. The ease of insertion coupled with no suture management or suture cutting provides increased tissue repairs, with increased strength and soft tissue (such as tendon) compression.

In an embodiment, a first tissue is approximated to a second tissue with a knotless, self-tensioning, self-locking surgical construct that includes two all-suture soft anchors connected by a fixed length of suture. The two soft anchors connected by suture are loaded onto a tip of an inserter such as a forked inserter. The soft anchors are inserted into tissue and, once the depth of insertion is achieved, the tissue being fixed pushes on the suture bridge so that the tension created causes the soft anchors to achieve a doughnut-like shape for fixation within the tissue. Alternative suture paths can form different securing shapes. Elastic suture can add compression. The steps can be repeated for additional soft anchors.

Methods of soft tissue repair which do not require tying of knots and allow tensioning at the time of the insertion are also disclosed. An exemplary method comprises inter alia the steps of: (i) providing a surgical construct formed of two soft anchors attached to a fixed length of a flexible coupler; and (ii) inserting the surgical construct into bone so that each of the two soft anchors changes its shape from a first configuration to a second configuration to create fixation during insertion, and to form a soft anchor locking “staple.” The method can further include (iii) tensioning the surgical construct during the insertion of the soft anchors, and without the need of additional tensioning steps. Tensioning occurs by the force of the tissue being fixed which pushes on the suture bridge in a direction different that the direction of the insertion of the soft anchors. The tissue force can push on the suture bridge in a direction opposite to the direction of the insertion of the soft anchors. The steps can be repeated to form additional soft anchor locking “staples,” depending on the extent of the repair and the surgeon's preference.

Referring now to the drawings, where like elements are designated by like reference numerals, FIGS. 1-10 illustrate surgical constructs 100, 100a (FiberTak® staple 100, 100a; FiberTak® soft anchor staple 100, 100a; construct 100, 100a) employed in exemplary method of tissue repairs 200, 300 (FiberTak® staple repair 200, 300; soft anchor locking staple repair 200, 300) of the present disclosure.

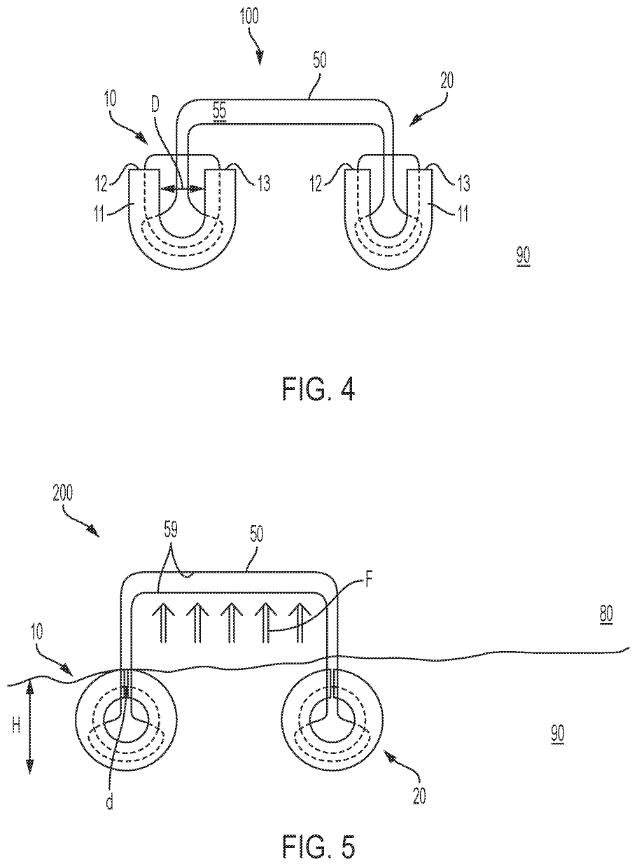

Surgical construct 100 of FIG. 1 includes two fixation devices 10, 20 connected by a fixed length of flexible coupler 50 (flexible strand 50). Each of the two fixation devices 10, 20 (shown in more detail in FIG. 4) can be in the form of a soft anchor (soft suture anchor, or all-suture soft knotless anchor) provided with a soft anchor sleeve 11 (sheath or tubular member 11) with two open ends 12, 13. At least one flexible coupler 50 extends through at least one region of each of the two fixation devices 10, 20. Flexible coupler 50 can enter the first tubular sheath of the first soft anchor 10 at a first location, extend along a longitudinal axis of the tubular sheath and then exit the first tubular sheath of the first soft anchor 10 at a second location; and then flexible coupler 50 can enter the second tubular sheath of the second soft anchor 20 at a first location, extend along a longitudinal axis of the tubular sheath and then exit the second tubular sheath of the second soft anchor 20 at a second location. Flexible coupler 50 can also enter and exit the first and second tubular sheaths of the first and second soft anchors 10, 20 through the open ends 12, 13 of each of the anchors 10, 20.

The flexible coupler 50 can extend through the sleeve (tubular sheath) of each fixation device 10, 20 in similar or different directions and/or orientations and/or locations. Details of an exemplary soft suture anchor with a soft anchor sleeve (sheath or tubular member) and flexible shuttling strands are set forth, for example in U.S. Pat. No. 10,849,734 issued Dec. 1, 2020, entitled “Methods of Tissue Repairs,” the disclosure of which is incorporated by reference in its entirety herein.

The flexible coupler 50 can include any flexible material, strand or ribbon such as suture or tape or combinations thereof, for example, multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The flexible coupler 50 can be also formed of suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein. The flexible coupler 50 can also be formed of an elastic material such as PEEK. Surgical constructs can be used with any type of flexible material, elastic material, or suture known in the art.

In an embodiment, flexible coupler 50 is formed of a single strand of material that is looped/joined to form loop 55 and optional knot 51. Loop 55 is a continuous, uninterrupted, flexible loop 55 with a fixed length and formed of a material such as suture. In one embodiment, the perimeter of loop 55 is fixed. In one embodiment, loop 55 can be formed of suture having a round cross-section. The suture can have the same or different diameters. Loop 55 can be also formed by splicing the flexible strand through itself, or by other methods known in the art, such as knotting, fusion, gluing, bonding, joining, braiding, interlinking, etc.

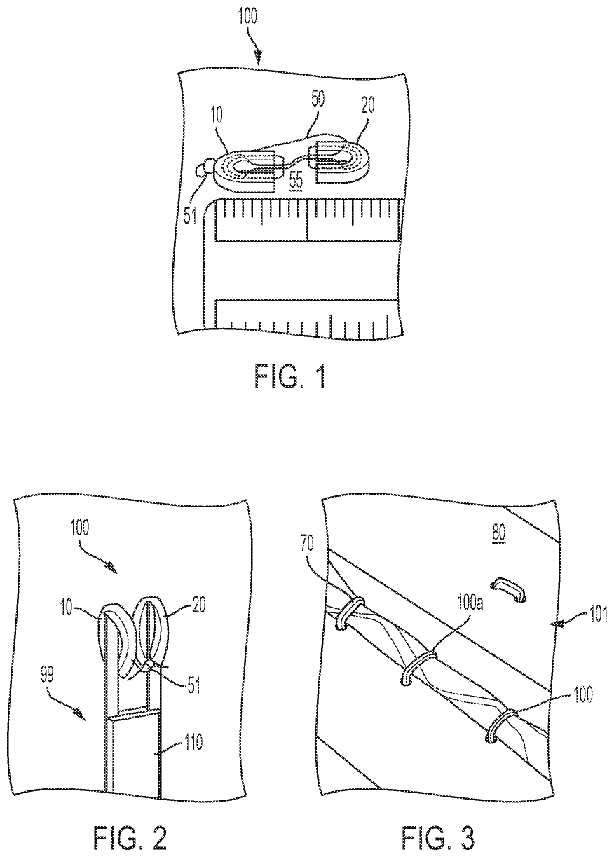

FIG. 2 illustrates surgical construct 100 loaded onto inserter 110 to form assembly 99. Inserter 110 can be a forked inserter to allow each of fixation device 10, 20 to rest and be secured to one of the two inserter tines. Assembly 99 allows the fixation device 10, 20 to be introduced into a first tissue 90 (bone 90), and to secure and tension a second tissue 80 (soft tissue 80) relative to first tissue 90, and as detailed below.

FIG. 3 illustrates a side-by-side comparison of two versions of the surgical constructs 100, 101 (Fibertak Staple concept) of the present disclosure, as well as a “hard” staple concept 70. The comparison shows the benefit with no material that could cause issue above the tissue 80 to be secured to another tissue, such as bone 90.

FIGS. 4 and 5 illustrate schematic side views of the surgical construct of FIG. 1 at the pre-insertion stage and at the post-insertion stage, respectively. Flexible coupler 50 extends within the two soft anchors 10, 20 and connects the two soft anchors 10, 20 therebetween.

At the pre-insertion stage of FIG. 4, the two soft anchors 10, 20 are relaxed, with the two open ends 12, 13 of each soft anchor located at a distance “D” from each other (i.e., the soft anchors 10, 20 have a first shape or configuration, about an U shape). At the post-insertion stage of FIG. 5, the two soft anchors 10, 20 are inserted into bone 90 so that the flexible coupler 50 rests upon soft tissue 80 to be fixated to bone 90. As the tissue force F of bridge 59 of the flexible coupler 50 pushes against the tissue 80, the soft anchor sleeve 11 (sheath or tubular member 11) of each of the two soft anchors 10, 20 becomes more rigid and achieves a “doughnut” shape for fixation (i.e., the soft anchors 10, 20 have a second shape or configuration). Once depth “H” is achieved within bone 90, the tissue being fixed pushes on suture bridge 59. That tension causes the soft anchors 10, 20 to form the “doughnut” shape for fixation, bringing the two open ends 12, 13 of each soft anchor closer, i.e., achieving a distance “d” between them which is smaller than the distance “D” at the pre-insertion stage.

Alternative suture paths of flexible coupler 50 connecting the two soft anchors 10, 20 can form different securing shapes for the soft anchors 10, 20 to be inserted into bone and secured to the bone. In addition, elastic suture can add compression to tissue 80. Regardless of the suture path or suture material, the surgical construct forms a soft tissue staple construct (formed by the two soft anchors 10, 20 connected by the suture 50) which is simple to insert and locks into place without secondary tensioning steps to achieve repair 200 (FIG. 5).

The prior art devices and known constructs have either tensionable constructs (finger trap or pre-tied knots or otherwise) or have no way of actively engaging to create fixation during insertion. The surgical constructs 100, 100a are soft anchors connected by a fixed length of suture loop 55 (no tensionable limb exiting) which by the nature of the path of the loop, causes the anchor to deform when the depth of insertion causes the suture tension to act.

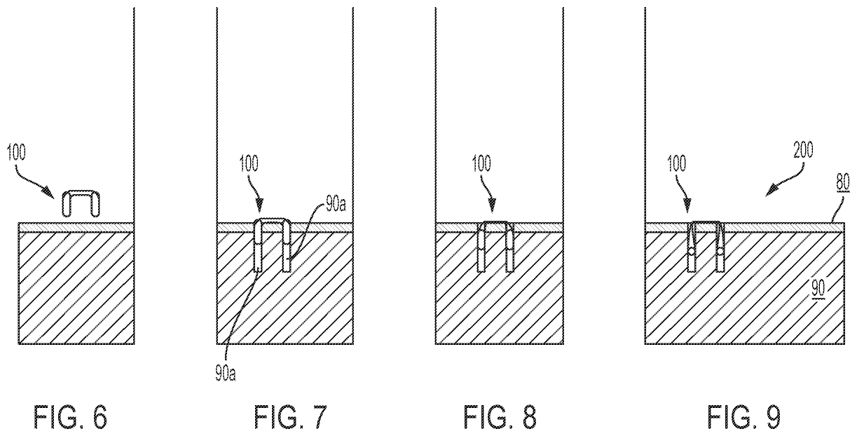

FIGS. 6-9 illustrate schematic, subsequent steps of tissue repair 200 to better illustrate tensioning of the repair at the time of insertion.

FIG. 6: FiberTak® staple 100 (surgical construct 100) is loose/relaxed on the inserter (not shown) and brought into proximity of second tissue 80 to be fixed/fixated to first tissue 90 (bone 90).

FIG. 7: Pilot holes 90a are prepared through tissue 80 and into bone 90. FiberTak® staple 100 begins to be tapped into place (into bone 90).

FIG. 8 illustrates the point at which sutures between the FiberTak® staple 100 contact second tissue 80 and begin to create tension in the construct.

FIG. 9: Compression of second tissue 80 causes the FiberTak® sheaths to be pulled into deformed shape (doughnut shape or second configuration) for fixation. Inserter can be removed and tissue 80 is fixed.

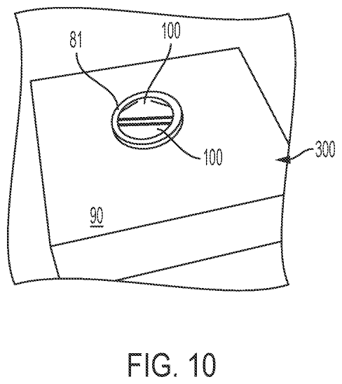

FIG. 10 illustrates exemplary repair 300 with two FiberTak® staples 100 that are fixing down an exemplary piece of silicone 81 instead of a tubular concept. Any number of FiberTak® staples 100 (surgical constructs 100) can be employed for attachment of a first tissue to a second tissue, for example, soft tissue 80 to bone 90. Any number of flexible couplers (such as flexible coupler 50) can be employed to connect the soft anchors 10, 20. If multiple couplers are employed, the couplers can be similar or different, and can be passed through and within the soft anchors 10, 20 through similar or different paths.

A surgical construct 100, 100a can include a fixed length of flexible coupler 50 (fixed length of a loop 55) with two or more soft anchors 10, 20 loaded onto, and connected by, the fixed length of flexible coupler 50. In an embodiment, two soft anchors 10, 20 are connected to a fixed length of flexible coupler 50. The flexible coupler 50 forms a flexible, continuous, uninterrupted loop 55 having a fixed length. The flexible, continuous, uninterrupted loop 55 can be knotless. The flexible, continuous, uninterrupted loop 55 can be knotted. The flexible coupler 50 can be suture. The flexible coupler 50 can be formed of an elastic material. The flexible coupler 50 can be elastic suture. The two or more soft anchors 10, 20 can be all-suture knotless anchors. The surgical construct 100, 100a can be employed in knotless fixation of tissue to tissue, for example, fixation of soft tissue 80 to bone 90. The surgical construct 100, 100a can consist essentially of a fixed length of flexible coupler 50 (fixed length of a loop) with two soft anchors 10, 20.

Methods of surgical tissue repairs are also disclosed. In an embodiment, a surgical construct 100, 100a provides knotless first tissue to second tissue fixation, without secondary tensioning steps, without any knot formation, with fewer passing steps, and with increased fixation and soft tissue compression. A surgical construct 100, 100a includes two soft anchors 10, 20 connected by a fixed length of a flexible coupler 50 (a fixed length of suture loop 55). The surgical construct 100, 100a is inserted into tissue 90 in a simple manner, to lock the construct into place and to tension the construct at the time of insertion, without necessitating any additional tensioning steps. The surgical construct 100, 100a creates a soft anchor locking “staple” constructed of soft anchors 10, 20 connected by the flexible coupler 50. The ease of insertion coupled with no suture management or suture cutting provides increased tissue repairs, with increased strength and soft tissue (such as tendon) compression.

In an embodiment, a second tissue 80 is approximated to a first tissue 90 with a knotless, self-tensioning, self-locking surgical construct 100, 100a that includes two all-suture soft anchors 10, 20 connected by a fixed length of suture 50. The two soft anchors connected by suture are loaded onto a tip of an inserter 110 such as a forked inserter 110. The soft anchors 10, 20 are inserted into tissue 90 and, once the depth of insertion is achieved, the tissue 80 being fixed pushes on the suture bridge 59 so that the tension created causes the soft anchors 10, 20 to deform and achieve a doughnut-like shape for fixation within the tissue 90. Alternative suture paths can form different securing shapes. Elastic suture can add compression. The steps can be repeated for additional soft anchors.

Methods of soft tissue repair 200, 300 which do not require tying of knots and allow tensioning at the time of the insertion are also disclosed. An exemplary method comprises inter alia the steps of: (i) providing a surgical construct 100, 100a formed of two soft anchors 10, 20 attached to and connected by a fixed length of a flexible coupler 50; and (ii) inserting the surgical construct 100, 100a into bone 90 so that each of the two soft anchors 10, 20 changes its shape from a first configuration to a second configuration to create fixation during insertion, and to form a soft anchor locking “staple.” The method can further include (iii) tensioning the surgical construct 100, 100a during the insertion of the soft anchors, and without the need of additional tensioning steps.

Tensioning occurs by the force of the tissue 80 being fixed which pushes on the suture bridge 59 in a direction different that the direction of the insertion of the soft anchors 10, 20. The tissue force can push on the suture bridge 59 in a direction opposite to the direction of the insertion of the soft anchors 10, 20 into bone 90. The steps can be repeated to form additional soft anchor locking “staples,” depending on the extent of the repair and the surgeon's preference.

Flexible coupler 50 (strand 50) can include a single filament, or fiber, or can include multiple continuous filaments, segments or regions of filaments that have different configurations (for example, different diameters and/or different compositions). The filament regions/segments may each be homogenous (i.e., formed of a same material) or may be a combination of homogenous and heterogenous (i.e., formed of a plurality of materials). Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible coupler can consist essentially of elastic material(s).

In an embodiment, flexible coupler 50 can be made of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The flexible strand can be also formed of suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein. Flexible coupler 50 can also include, and be manufactured with, any kind of material (suture, nylon, silk, UHMWPE. metal, bioabsorbable, etc.) that can allow the flexible strand to form a loop.

Flexible coupler 50 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture, loop security, pliability, handleability or abrasion resistance, for example.

Flexible coupler 50 can be also provided with tinted tracing strands, or otherwise contrast visually with other areas/regions of the construct, which remains a plain, solid color, or displays a different tracing pattern, for example. Various structural elements of flexible coupler 50 such as loop 55 and/or bridge 59 may be visually coded, making identification and handling of the suture simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical construct consisting of a fixed length of flexible coupler connected to a first soft anchor and to a second soft anchor.

2. The surgical construct of claim 1, wherein the fixed length of flexible coupler forms a staple across tissue to be fixated with the first soft anchor and the second soft anchor.

3. The surgical construct of claim 1, wherein the surgical construct is self-tensioning.

4. The surgical construct of claim 1, wherein the fixed length of flexible coupler is secured to the first soft anchor and to the second soft anchor by a knot.

5. The surgical construct of claim 1, wherein the fixed length of flexible coupler passes at least once through a first tubular sheath of the first soft anchor and at least once through a second tubular sheath of the second soft anchor to form a closed, continuous, flexible loop with a fixed perimeter.

6. The surgical construct of claim 5, wherein the fixed length of flexible coupler enters the first tubular sheath at a first location, extends within the first tubular sheath and exits the first tubular sheath at a second location, wherein the second location is different from the first location.

7. The surgical construct of claim 6, wherein the fixed length of flexible coupler enters the second tubular sheath at a first location, extends within the second tubular sheath and exits the second tubular sheath at a second location, wherein the second location is different from the first location.

8. The surgical construct of claim 1, wherein each of the first soft anchor and the second soft anchor is an all-suture anchor, and the flexible coupler is round suture.

9. A surgical construct comprising:
a first soft anchor with a first flexible sheath having a first open end and a second open end;
a second soft anchor with a second flexible sheath having a first open end and a second open end; and
a flexible coupler connecting the first soft anchor to the second soft anchor, wherein the flexible coupler has a fixed length and wherein the flexible coupler forms at least one closed, flexible, continuous loop by being passed at least once through the first flexible sheath of the first soft anchor and at least once through the second flexible sheath of the second soft anchor.

10. The surgical construct of claim 9, wherein the first flexible sheath is a first suture tube, the second flexible sheath is a second suture tube, and the flexible coupler is round suture.

11. The surgical construct of claim 9, wherein the first soft anchor and the second soft anchor reside into bone and wherein a portion of the flexible coupler forms a bridge over tissue to be secured to the bone.

12. The surgical construct of claim 11, wherein the tissue is soft tissue.

13. The surgical construct of claim 11, wherein the tissue is tendon, ligament or graft.

14. The surgical construct of claim 9, wherein the flexible coupler is formed of suture, silk, cotton, nylon, polypropylene, polyethylene, ultrahigh molecular weight polyethylene (UHMWPE),
polyethylene terephthalate (PET), polyesters or copolymers, or combinations thereof.

15. The surgical construct of claim 9, wherein the flexible coupler consists essentially of elastic material.

16. A surgical repair comprising:
a first soft anchor secured into a first tissue at a first location and a second soft anchor secured into the first tissue at a second location; and
a suture bridge attached to the first soft anchor and to the second soft anchor and extending above a second tissue.

17. The surgical repair of claim 16, wherein the first tissue is bone and the second tissue is soft tissue to be attached to bone.

18. The surgical repair of claim 16, wherein the suture bridge has a fixed length and forms a locking staple with the first and second soft anchors.

19. The surgical repair of claim 16, wherein at least one of the first and second soft anchors secured into the first tissue has a doughnut-like shape.

20. A method of self-tensioning tissue repair comprising:
inserting through a first tissue and through a second tissue a first soft anchor and a second soft anchor, wherein the first soft anchor is connected to the second soft anchor by a flexible coupler with a fixed length; and
securing, in the second tissue, the first soft anchor at a first location and the second soft anchor at a second location.

21. The method of claim 20, wherein securing the first soft anchor and the second soft anchor is conducted simultaneously with tensioning of the repair, and without requiring any additional tensioning.

22. The method of claim 20, further comprising:
passing at least once the flexible coupler through a first tubular sheath of the first soft anchor;
subsequently, passing at least once the flexible coupler through a second tubular sheath of the second soft anchor to form a flexible, closed, continuous loop with a fixed perimeter connecting the first soft anchor to the second soft anchor;
securing the first soft anchor and the second soft anchor to a forked inserter;
passing the first soft anchor and the second soft anchor through the first tissue and through the second tissue; and
securing simultaneously the first soft anchor into a first opening in the second tissue at the first location, and the second soft anchor into a second opening in the second tissue at the second location, to allow tensioning of the repair over the first tissue without additional tensioning steps.

23. The method of claim 22, wherein the flexible, closed, continuous loop extends over a surface of the first tissue and allows the first tissue to achieve a desired tension relative to the second tissue.

24. The method of claim 20, wherein the first tissue is tendon, ligament or graft and the second tissue is bone.

25. The method of claim 20, wherein each of the first and second soft anchors is an all-suture anchor formed of a single suture material, and wherein the flexible coupler is elastic suture.
